(19) United States
(12) Patent Application Publication
(10) Pub. No.: US 2004/0106907 A1
(43) Pub. Date: Jun. 3, 2004

(54) Device for sucking body and method thereof

(76) Inventor: Shih-Ta Liu, Taoyuan City (TW)

Correspondence Address: HAVERSTOCK & OWENS LLP, 162 NORTH WOLFE ROAD, SUNNYVALE, CA, 94086, US

(21) Appl. No.: 10/677,889

(22) Filed: Oct. 1, 2003

(30) Foreign Application Priority Data
Oct. 2, 2002 (TW) 91122785
Oct. 2, 2002 (TW) 91122786
Oct. 2, 2002 (TW) 91122787

(51) Int. Cl. A61M 1/00

(52) U.S. Cl. 604/313; 604/74; 604/46; 601/6

(57) ABSTRACT

A method for sucking a body through a sucking device, in which the sucking device includes a vacuum generator, a reservoir, a shield having at least one opening, a first tube and a second tube. The first tube communicates the shield with the vacuum generator and the second tube communicates the shield with the reservoir. The method comprising steps of: covering the shield on an area of the body; generating a negative pressure in the shield by the vacuum generator so as to gradually bulge the body area; and filling a filling medium into the shield from the reservoir for decreasing the negative pressure so as to restore the body area.

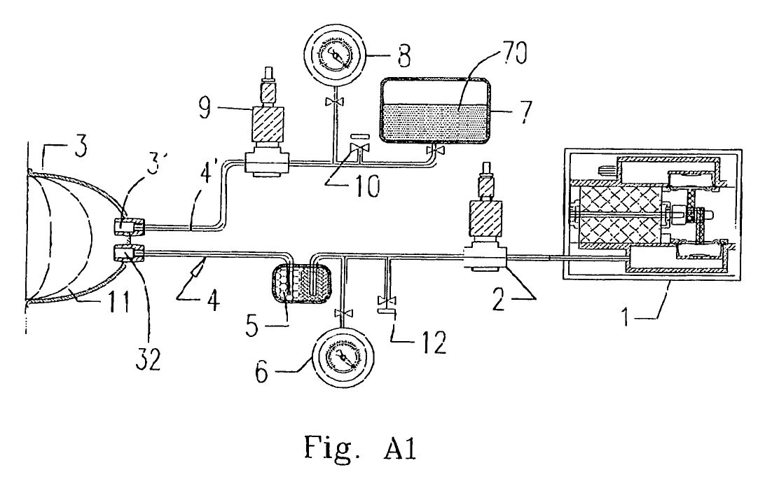

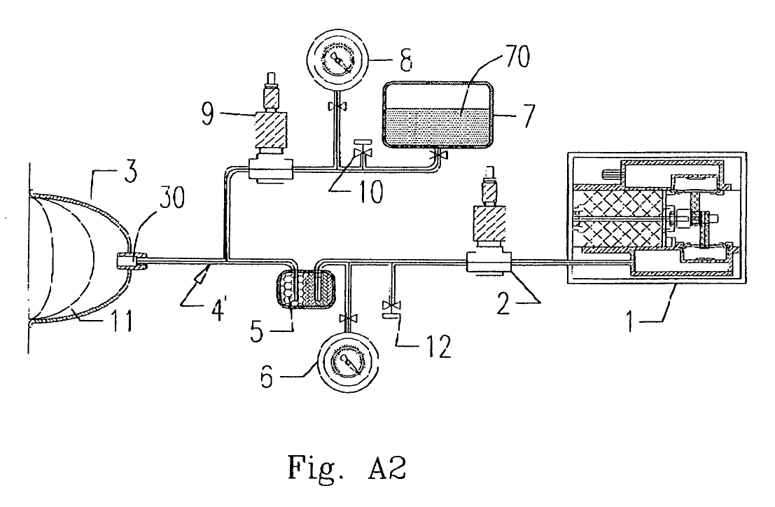

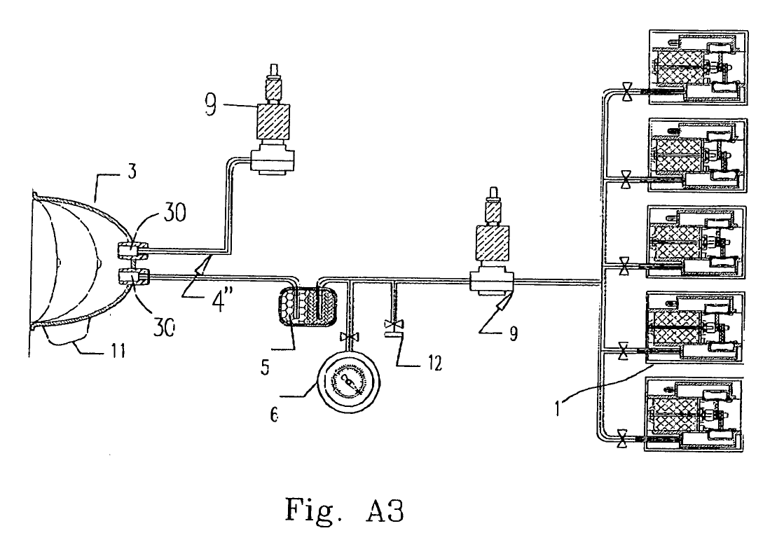

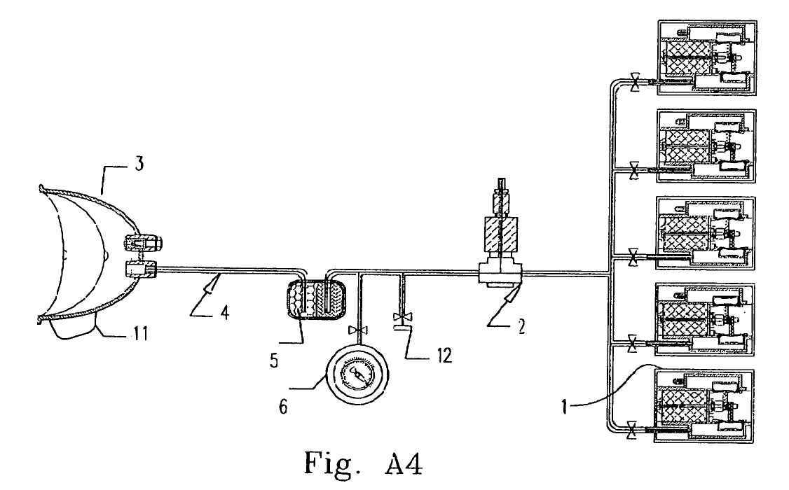

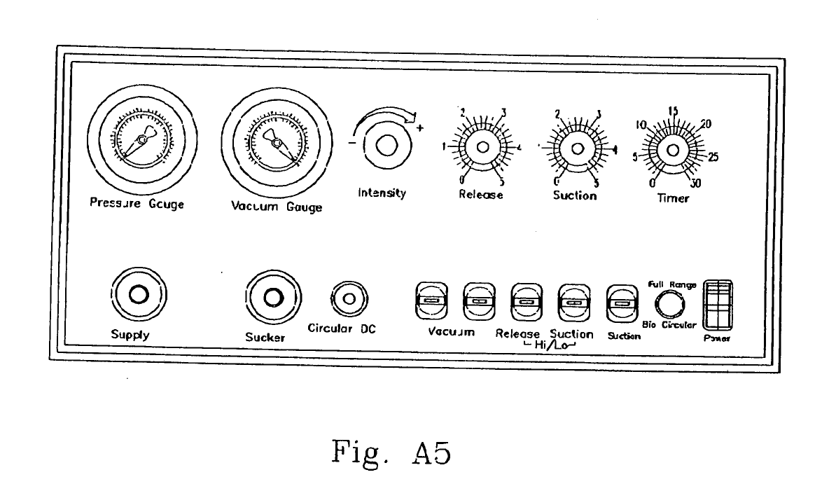

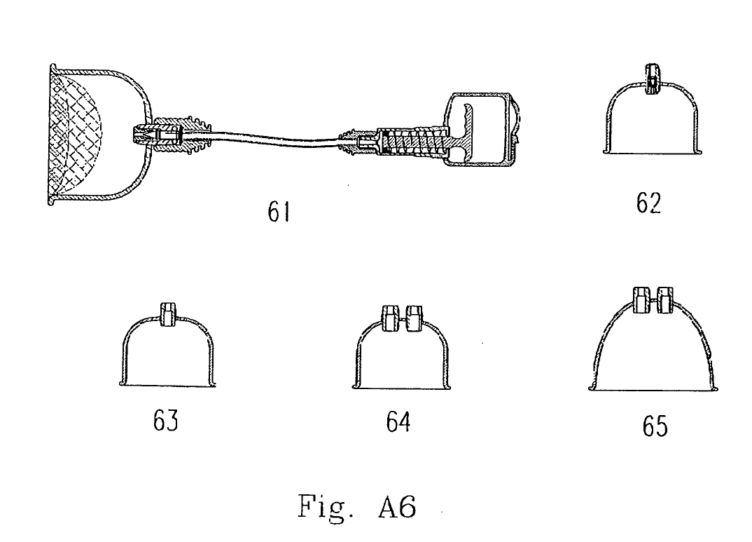

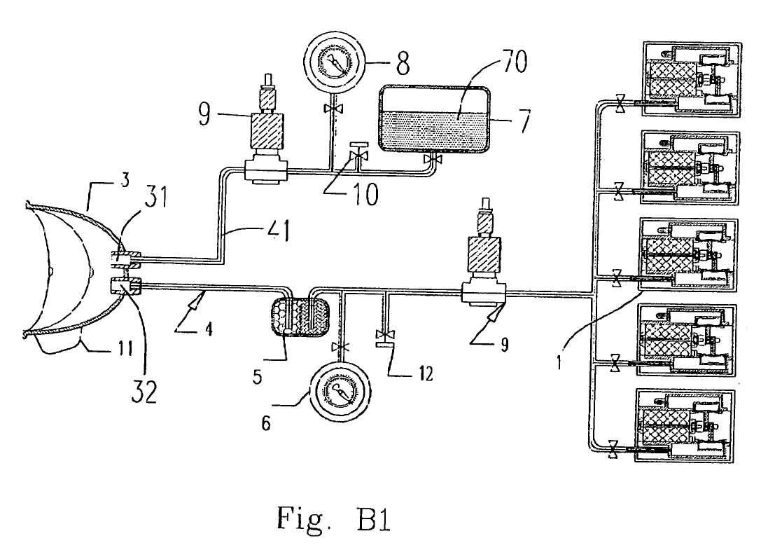

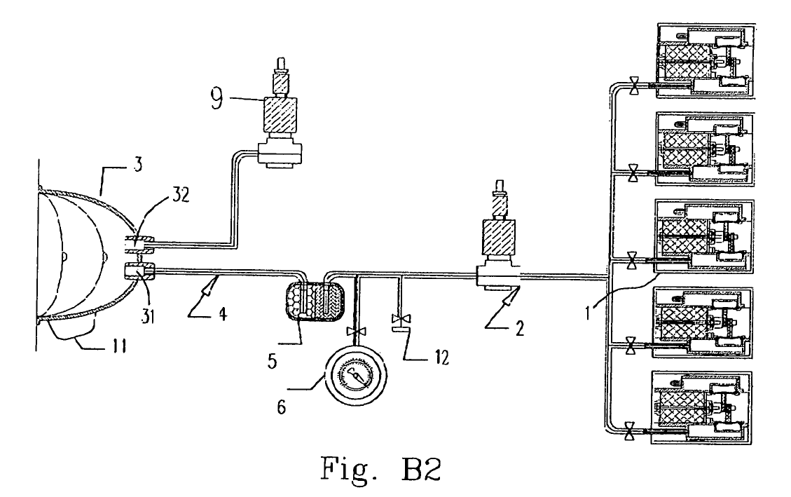

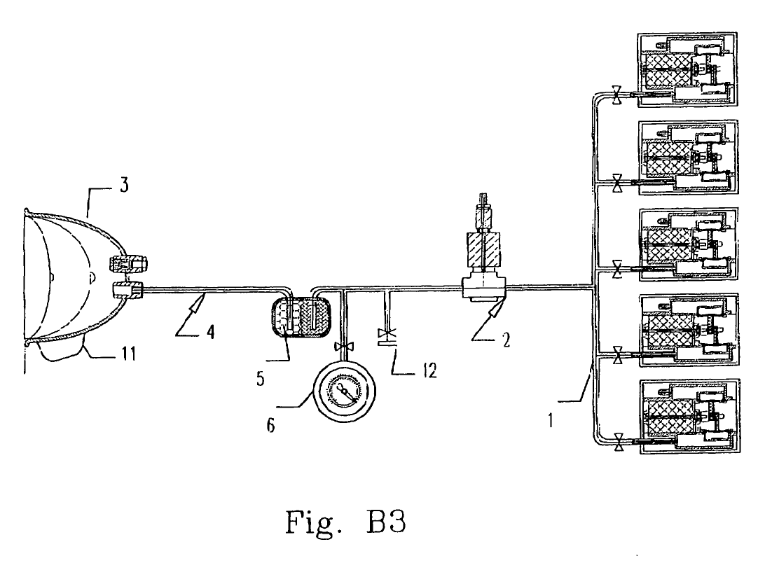

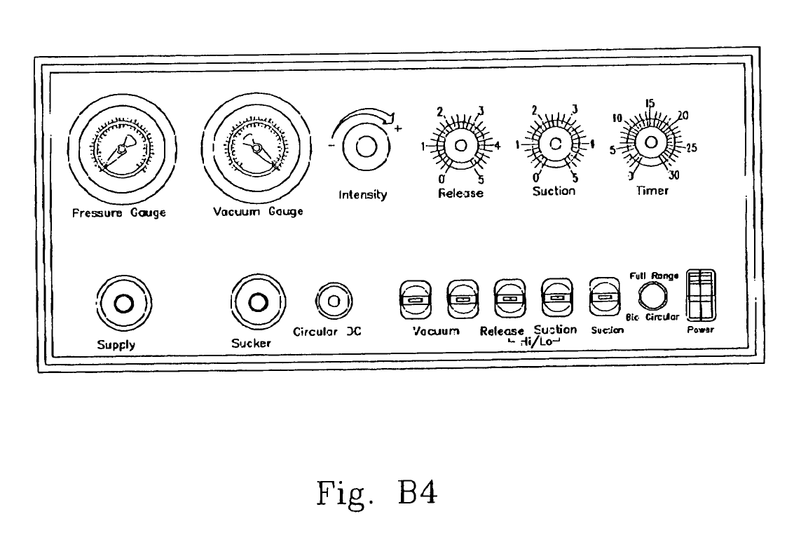

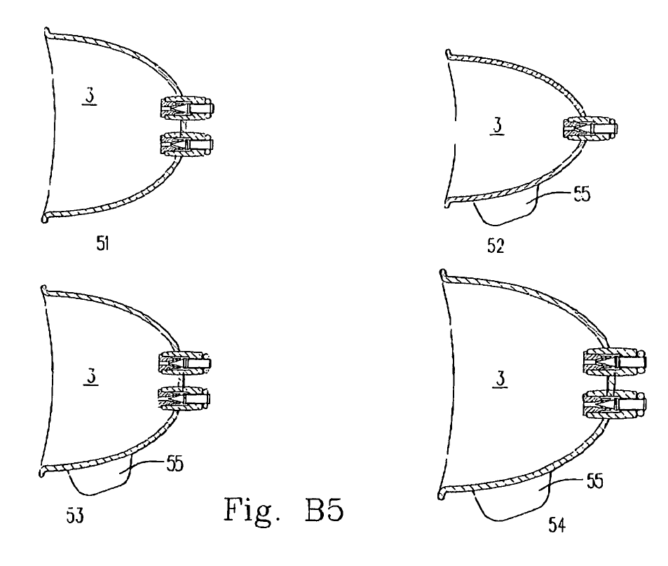

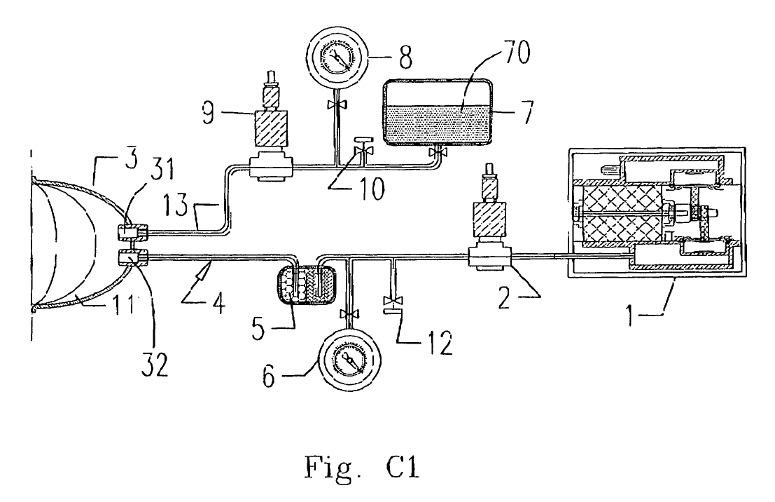

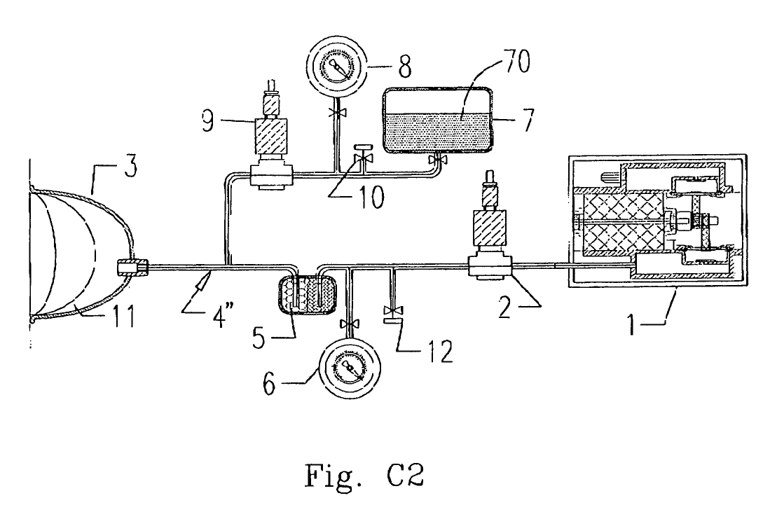

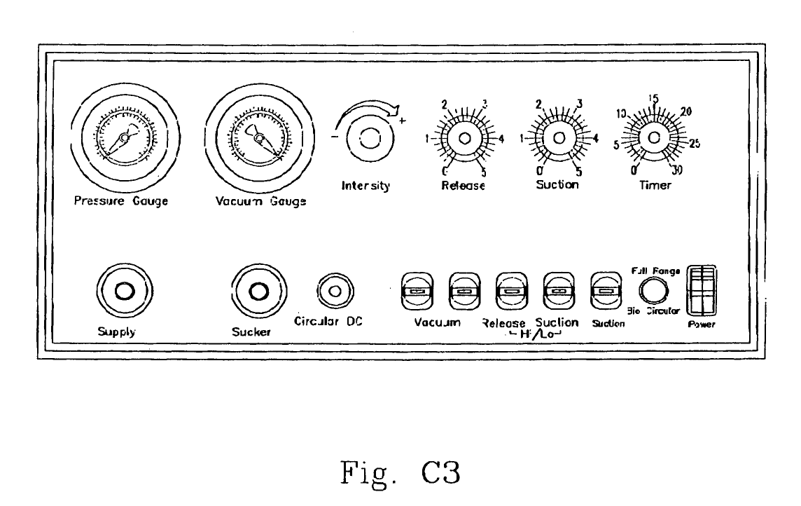

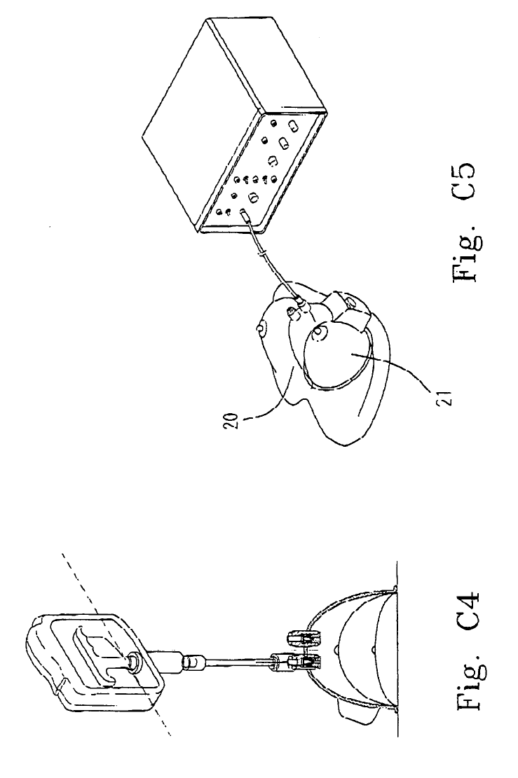

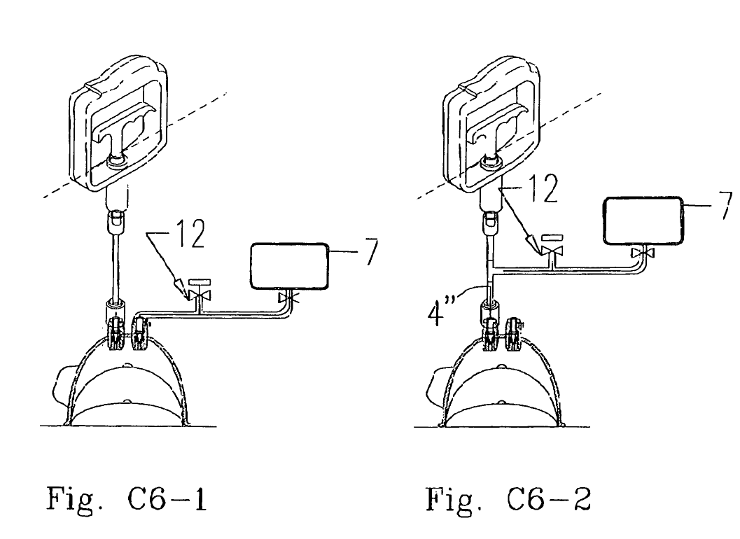

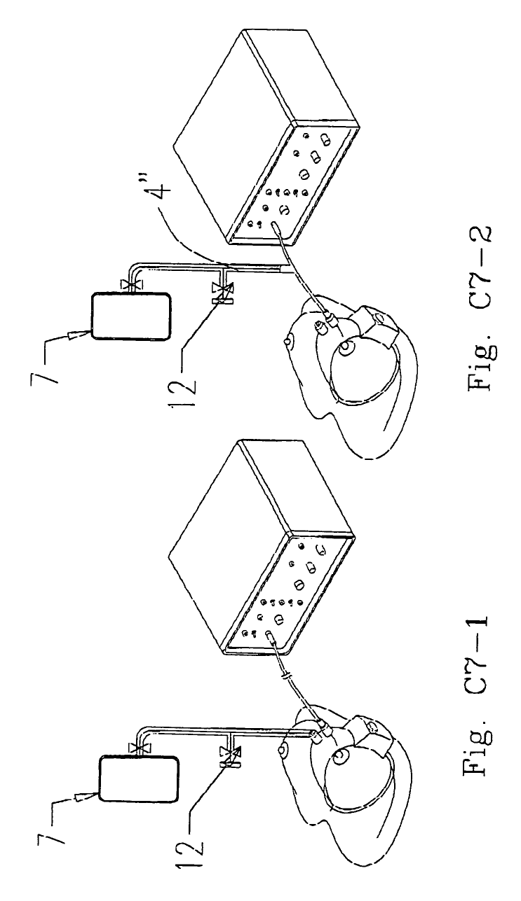

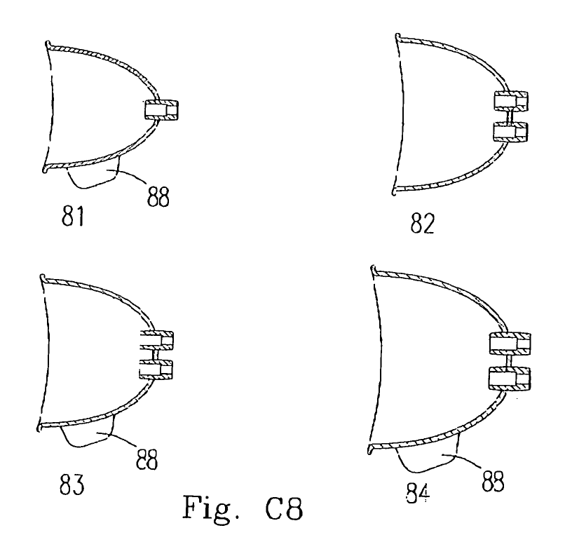

### FIELD OF THE INVENTION

The present invention is related to a device and a method for sucking a body, and more particularly, to the device and method for use in a can sucking therapy, aspiration of ichors or sanies and breast augmentation.

### BACKGROUND OF THE INVENTION

Cupping is an ancient Chinese medical method with thousand-years history. Generally, a partial vacuum is created in cups placed on the skin either by means of heat or suction. This draws up the underlying tissues and causes local congestion. When the cup is left in place on the skin for a few minutes, blood stasis is formed and localized healing takes place.

Nowadays, the cupping therapy has been a very important part of the traditional medical science. And there are various type of devices in the market for sucking a body and achieving the purpose of cupping therapy. Even more, those device can massage the breast and draw the prior from skin Conventionally, the foregoing purposes are achieved by the prior arts shown below.

In cupping therapy, traditionally the cup could be horn, bamboo can, pottery can, metallic can and glass can. Now, the cup is mostly made of acrylic In order to generate a negative pressure in a can for sucking on a body, the artemisia is burned in a can before placed on the skin at the ancient time. Now, a pump or an electric pump is mostly used.

A well known conventional cupping therapy device has a vacuum mechanism for producing a negative pressure, plural cups with several sizes and a tube for communicating the vacuum mechanism and the cups.

The common steps for the conventional method of the cupping therapy are as follows. First, place the cup is placed on an area of a body after the vacuum mechanism is communicated with the cup. Secondly the air sucked out from the cup to draw the body up beneath the cup. Thirdly, the cup is took off from the body after ten minutes around. However, the conventional method has some defects. One is the sucked skin area is easily to be seriously congested, and the other is the sucked skin area is forced to move and thereby the blood circulation is temporary interrupted. Therefore, the effectiveness is affected.

With regard to aspirating ichors and sanies from breast, traditionally it needs needle-aspirating therapy or the operation to resect partial tissue in breast. Those disease such as breast cyst near mammary gland, which usually appears after the breast-feeding is stopped. The mammary gland is full of milk and dead cells, and patient feels hurt when the breast is touched. While the ichors or sanies start bleeding out from the breast, it is necessary to use the needle to draw the ichiors out or have an operation to resect the cyst in order to prevent the aggravation of the situation.

Another breast disease is acute mastitis. The acute-mastitis is cause by of the breast infection, which results in the breast inflammation, swelling and pain. Sometimes this disease can be controlled by antibiotic. But sometimes it needs to use the needle to aspirate the ichors or cut an opening for the ichors/sanies to flow out.

In the conventional therapy for the easily-recurrent disease, medical doctors usually advise patient to take antibiotics to control the disease, observe whether the situation is going bad, and the consider an operation. However, there is no other auxiliary treatment to help control these kind of diseases. Therefore, the present invention is provided as an auxiliary treatment. The patient may use the present invention to draw the ichors/sanies out from the breast without any operations.

With regard to the breast augmentation, the conventional method is to massage the breast by pumping the air in and out of a can and vibrating the breast. During a cycle of pumping, the conventional method always pumps the fluxed air back into the can. However, the refluxing air always contains impure or contaminated gas, which is the biggest disadvantage when the conventional cupping therapy device is used for breast augmentation.

In the present market, the conventional breast augmentation devices are low-frequency vibrator, and cupping device modified by enlarging the cup of the cupping therapy device. One type of the cupping device keeps the breast at a static position after sucking the breast up. Another breast augmentation device, which pumps the air in and out, moves the breast up and down, which is believed more effective than the previous type. The present invention further provides a cup sucking device with a vibrator to produce a three-dimensional vibration, which is better than the only up and down motions. Thus, for reinforcing the efficiency of the breast augmentation mechanism, a advanced device for sucking a body is provided.

### SUMMARY OF THE INVENTION

The present invention provides a device for sucking a body. It includes comprises a vacuum generator for providing a negative pressure; a reservoir for providing a filling medium stored therein; a shield for covering an area of the body; a first tube communicating the shield with the vacuum generator; and a second tube communicating the shield with the reservoir.

The device as set forth above, the shield further includes at least one opening for communicating the first and second tube.

The device as set forth above, the first tube and the second tube are combined as a combination tube to be connected to the at least one opening.

The device as set forth above, the first tube and the second tube are communicated with the shield through a connector.

The device as set forth above, the vacuum generator is selected from a group consisting of a manual air pump, an electrical air pump, and a vacuum pump.

The device as set forth above the device has multiple vacuum generators.

The device as set forth above, the reservoir has a valve for controlling a communication of the second tube.

The device as set forth above, the first tube connects to a controller for controlling timings of a setup, a generation, and a release of the negative pressure.

The device as set forth above, the filling medium is one of a gas and a liquid.

The device as set forth above, the filling medium is provided from one of a can and a supplying tube.

The device as set forth above, the shield is selected from various standards.

The device as set forth above, a number of the opening is one of one and two.

The device as set forth above, the shield further including a vibrating mechanism.

The present invention provides a method for sucking a body through a sucking device, wherein the sucking device includes a vacuum generator, a reservoir, a shield having at least one opening, a first tube and a second tube, the first tube communicates the shield with the vacuum generator and the second tube communicates the shield with the reservoir. The method includes steps of first, covering the shield on an area of the body; second, generating a negative pressure in the shield by the vacuum generator so as to gradually bulge the body area; and third, filling a filling medium into the shield from the reservoir for decreasing the negative pressure so as to restore the body area.

Preferably, the first tube and the second tube are communicating with the at least one opening.

Preferably, the vacuum generator is selected from a group consisting of a manual air pump, an electrical air pump; and a vacuum pump.

Preferably, the first tube further connects to a controller for controlling timings of a setup, a generation, and a release of the negative pressure.

Preferably, the filling medium is one of a gas and a liquid

Preferably, a number of the opening is one of one and two.

Preferably, the method further has a stop of vibrating tile shield by a vibrating mechanism connected thereon.

The foregoing and other features and advantages of the present invention will be more clearly understood through the following descriptions with reference to the drawings, wherein:

### BRIEF DESCRIPTION OF THE DRAWINGS

FIGS. A1 to A4 are schematic views showing the cupping therapy device according to the present invention;

FIG. A5 is a schematic view showing the control panel according to the present invention;

FIG. A6 is a section view showing various shields accommodating to the device of the present invention;

FIGS. B1 to B3 are schematic views showing the cupping therapy device according to another the embodiment of the present invention;

FIG. B4 is a schematic view showing the control panel according to the present invention;

FIG. B5 is a schematic view showing the various shields further connecting to a vibrator.

FIGS. C1 and C2 are schematic views showing the breast augmentation device according to another embodiment of the present invention;

FIG. C3 is a schematic view showing the control panel according to the present invention;

FIG. C4 is a schematic view showing a device with a manual air pump covering on a human breast according to the present invention;

FIG. C5 is a schematic view showing a device with an electrical air pump covering on a human breast according to the present invention;

FIG. C6 is a schematic view showing a device with a manual air pump and a reservoir according to the present invention;

FIG. C7 is a schematic view showing a device with an electrical air pump and a reservoir according to the present invention; and

FIG. C8 is a section view showing various shields with different sizes according to the present invention.

### DETAILED DESCRIPTION OF THE PREFERRED EMBODIMENT

It is an object of the present invention is to provide a device for sucking body to intermittently suction, aspirating ichors, and breast augmentation.

The present invention will now be described more specifically with reference to the following embodiments for different situations. The device provided in the present invention can be used for different purposes. The first situation is used for cupping therapy. The device for sucking a body includes a vacuum generator, a shield for covering an area of body, and a reservoir for providing a filling medium into the shield. The biggest difference between the prior art and the present invention is that the device of the present invention intermittently sucks an area of a body, that produces a intermittent suction. However, the prior device continuously sucks the skin, which blocks the blood flow too long.

Please refer to the FIGS. A1 to A4, which shows the various embodiments of cupping therapy device according to the present invention. In the FIG. A1, the shield 3 has two openings 31, 32 and a fast cupping therapy effect can be achieved. When a vacuum generator 1 activates, the shield 3 sucks on an area of a body 11. Because of the generated negative pressure in the shield 3, the area of the body 11 is bulged. When the vacuum generator 1 stops generating the negative pressure, the body 11 stops bulging up. When the negative pressure is released, the body 11 is retrieved. By repeating the cycle, a fast cupping therapy effect is achieved. The efficiency of promoting blood circulation by intermittent suction is much higher than the prior device.

Please refer to the FIG. A1. The vacuum generator 1 includes one or multiple vacuum pumps. The first valve 2 controls whether the suction force from the vacuum generator 1 reaches to the shield 3 through the first tube 4 or not. The first adjustable valve 12 adjusts the suction force generated from the vacuum generator 1. Generally speaking, the greatest negative pressure is up to −46.7 kPa (−350 mmHg) so as to prevent from hurting the body 11. The vacuum gauge 6 shows the vacuum degree in the shield 3. The filter 5 is used for filtering the impurities from the filling medium 70 or the air from the atmosphere. The first tube 4 communicates the vacuum generator 1 with the shield 3. The second valve 9 controls whether the filling medium 70 flows into the shield 3 through the second tube 41 or not. The filling medium 70 is used for decreasing the negative pressure and retrieving the body 11 covered by the shield 3. The reservoir 7 has the filling medium 70 (a gas or a liquid) filled therein to substitute the normal air provided for the shield 3. The filling medium 70 is curative. The pressure gauge 8 shows the pressure in the reservoir 7. The second adjustable valve 10 adjusts the quantity of the filling medium 70 flowed out from the reservoir 7 so as to control the speed of the retrieving of body 11.

Please refer to FIG. A3. The shield 3 has only one opening 30. Both of the suction force and filling the filling medium 70 are through the only one opening 30 and a combination tube 4′.

Please refer to FIG. A3. This embodiment has plural vacuum generators 1. And there is no reservoir communicating with the shield 3. The valve 9 controls the air of the atmosphere flowed into the shield 3 to decrease the negative pressure therein so as to control the retrieving of the body 11. By repeating the cycle of air sucking and refluxing, the cupping device of the present invention reaches a fast and efficient cupping therapy effect.

Please refer to FIG. A4. This embodiment has plural vacuum generators 1. Their is neither reservoir nor the valve 9 communicating with the shield 3. The ambient air directly reflows to the shield 3 through the first valve 2. So as to control the retrieving of the body 11. By repeating the cycle of air sucking and reflux, the cupping therapy device of the present invention reaches a fast cupping therapy effect.

Please refer to FIG. A5, which shows the control panel to control the timing of the whole schedule for a cupping therapy. The suction knob sets up the scale of the negative pressure applied to the body. The release knob sets up the scale of the releasing of the negative pressure. The pressure gauge shows the pressure inside the reservoir 7(FIG. A2). The knob “Power” is a switch for conducting an electric power. The sign “Sucker” is a communicating opening for connecting the shield 3 with vacuum generator 1. The sign “Supply” is a communicating tube for connecting the shield 3 with the reservoir 7. The panel further has timer and controller for many kinds of setting.

For dealing with many situations of body, the scale of the negative pressure and the timing to generate and release the negative pressure can be set up and adjusted. The blood circulation is promoted by the intermittent suction because the cupping therapy brakes the blockage in the blood. Therefore, the curative effect of cupping therapy can be very successful.

In the whole therapy, the reservoir 7 is filled with the curative fluid that flows into the shield 3 instead of the air. The reservoir 7 is a tank or a pipeline to transfer the curative fluid and therefore increase the effect of the cupping therapy to have a special medical effect.

Please refer to the FIG. A6. Various sizes of the shields are shown. Shield 61 shows the conventional shield connected with prior sucking device for cupping therapy. The shield 62 is a shield with conventional standard for cupping therapy. The shield 63 is a shield having one opening in the present invention. The shields 64 and 65 show the shields having two openings in the present invention. One of the openings is for suction. The other is for air reflux to reduce the negative pressure, or filling the filling medium into the shield to reduce the negative pressure.

The conventional method of the cupping therapy are as follows. First, the cup is placed on an area of a body after the vacuum mechanism is communicated with the cup. Secondly, the air is sucked out from the cup to draw the body up beneath the cup. Thirdly, the cup is took off from the body after ten minutes around. However, the conventional method has some defects. One is the sucked skin area is easily to be over-congested, and the other is the sucked skin area is forced to move and thereby the blood circulation is temporary interrupted. Therefore, the effectiveness is affected. On the contrary, the cupping therapy device of present invention is intermittent suction, which makes the body bulged and released repeatedly. A better blood circulation is forcibly produced and thereby it is more effective than the conventional one.

Furthermore, the reservoir of the present invention provides a clean air or a special curative gas flowed into the shield. The reservoir is also the supplier of the curative liquid. By the adoption of the reservoir, the cupping therapy becomes more effective.

The second situation is using the cupping therapy device to aspirate the ichors/sanies out from breast. The present invention provides a device and method to suck out the ichors to help cure the breast disease. A vacuum environment is created to suck out the ichors from breast through endocrine gland, mammary gland and pores on skin.

Please refer to the FIG. B1. It is an embodiment of the present invention to provide a device and method for sucking out the ichors from breast without any surgery. This method helps cure the breast disease such as mastitis.

After a shield 3 covers on a breast 11, a negative pressure is created in the shield 3 for drawing the breast 11 up. When the negative pressure is decreased, the breast 11 is retrieved to original shape. Therefore, by repeating the drawing up and retrieving the breast 11, the ichors will be gradually sucked out from the breast 11. And the breast cyst will become smaller or even disappeared when the cause is simply blood blockage.

In the FIG. B1, the vacuum generator 1 includes one or more vacuum pumps. The first valve 2 controls whether the suction force from the vacuum generator 1 reaches the shield 3 through the first tube 4 or not. The first adjustable valve 12 adjusts the suction force generated from the vacuum generator 1. Generally speaking, the greatest negative pressure is up to −46.7 kPa (−350 mmHg) so as to prevent from hurting the breast 11. The vacuum gauge 6 shows the vacuum degree in the shield 3. The filter 5 is used for filtering the impurities from the filling medium 70 or the air from the atmosphere. The first tube 4 communicates the vacuum generator 1 with the shield 3. The second valve 9 controls whether the filling medium 70 flows into the shield 3 through the second tube 41 or not. The filling medium 70 is used for decreasing the negative pressure and retrieving the breast 11 covered by the shield 3. The reservoir 7 has the filling medium 70 (a gas or a liquid) therein to substitute the normal air provided for the shield 3. The filling medium 70 is curative. The pressure gauge 8 shows the pressure in the reservoir 7. The second adjustable valve 10 adjusts the quantity of the filling medium 70 flowed out from the reservoir 7 so as to control the speed of the retrieving of breast 11.

Please refer to FIG. B2. It shows the another embodiment of the present invention. There is no reservoir communicating with the shield 3. The valve 9 controls the air of the atmosphere flowed into the shield 3 to decrease the negative pressure therein, so as to control the retrieving of the breast 11. By repeating the cycle of air sucking and reflux, the cupping therapy device of the present invention can suck out the ichors from the breast 11.

Please refer to FIG. B3. This embodiment has plural vacuum generators 1. And there is neither reservoir nor the valve 9 communicating with the shield 3. The air of the atmosphere is reflows to the shield 3 through the first valve 2 so as to control the retrieving of the breast 11. By repeating the cycle of air sucking and reflux, the cupping therapy device of the present invention can suck out the ichors from the breast 11.

Please refer to FIG. B4, which shows the control panel to control the timing of the whole schedule for sucking out the ichors. The timer knob sets up the time of the schedule of therapy. The suction knob sets up the scale of the negative pressure applied to the breast 11. The release knob sets up the scale of the releasing of the negative pressure. The intensity knob sets up the biggest negative pressure in the whole schedule. The vacuum gauge shows the scale of the negative pressure. The pressure gauge shows the pressure inside the reservoir 7(FIG. A2). The knob “Power” is a switch for conducting an electric power. The sign “Sucker” is a communicating opening for connecting the shield 3 with the vacuum generator 1. The sign “Supply” is a communicating tube to the shield 3 with the reservoir 7. The panel further has timer and controller for many kinds of setting.

In the whole schedule, the reservoir 7 is filled with the curative filling medium 70 to replace the air provided for the shield 3 so as to have a special medical effect.

Please refer to the FIG. B5. Various sizes of the shields are shown. Shield 51 is a shield without vibrator 55. Shields 52, 53 and 54 show each of shields in various sizes, which all have a vibrator 55 to enforce the effect of therapy.

Therefore, the device provided in the present invention can suck out the ichors from breast so that the repeated suction helps cure the mastitis and mammary gland cyst. Therefore, the need for breast sugary is reduced.

In the situation of breast augmentation, the present invention provide a device and method for breast augmentation. The filling medium is a clean gas or a specific fluid. Furthermore, a vibrator can be added in the device of the present invention to increase the effect of breast augmentation.

Please refer to the FIG. C1, which shows a device for breast augmentation by using a shield with two openings and a reservoir to increase the effectiveness and safety of the device.

According to the FIG. C1. When a vacuum generator 1 activates, the shield 3 sucks on a breast 11. Because of the generated negative pressure in the shield 3, the breast 11 is bulged. When the vacuum generator 1 stops generating negative pressure a filling medium 70 is provided from a reservoir 7 and flowed into the shield 3 through a tube 13, the negative pressure will be decreased, and thereby the breast 11 is retrieved. By repeating the cycle the breast is massaged more efficiency and the effect of breast augmentation is better.

The biggest difference between the present invention and the prior art is that the present invention further provides a reservoir 7 to provide a clean air or a filling medium 70 flowed into the shield 3. Another the other difference is that the shield 3 of the present invention has two openings. One communicates with the vacuum generator 1, and the other communicated with the reservoir 7.

In the FIG. C1, the vacuum generator 1 is selected from a group consisting of a manual air pump, an electrical air pump, and a vacuum pump, which can be single or multiple when embodied. The first valve 2 controls whether the suction force from the vacuum generator 1 reaches to the shield 3 through the first tube 4 or not. The first adjustable valve 12 adjusts the suction force generated from the vacuum generator 1. Generally speaking, the greatest negative pressure is up to −16.7 kPa (−350 mmHg) so as to prevent from hurting the breast 11. The vacuum gauge 6 shows the vacuum degree in the shield 3. The filter 5 is used for filtering the impurities from the filling medium 70 or the air from the atmosphere. The first tube 4 communicates the vacuum generator 1 and the shield 3. The second valve 9 controls whether the filling medium 70 flows into the shield 3 through the tube 13 or not. The filling medium 70 is used for decreasing the negative pressure and retrieving the breast 11 covered by the shield 3. The reservoir 7 has the filling medium 70 (a gas or a liquid) filled therein to substitute the normal air provided for the shield 3. The filling medium 70 is curative. The pressure gauge 8 shows the pressure in the reservoir 7. The second adjustable valve 10 adjusts the quantity of the filling medium 70 flowed out from the reservoir 7 so as to control the retrieving speed of the breast 11. The second tube 13 communicates the reservoir 7 with the shield 3.

Please refer to the FIG. C2, which shows another embodiment of the present invention. The vacuum generator 1 and the reservoir 7 both communicate with the shield 3 through one combination tube 4″. The reservoir 7 stores the filling medium 70, which is a clean air or specific fluid. The filling medium 70 flows into the shield 3 to decrease the negative pressure generated therein and then retrieve the breast 11 to original shape. By drawing up and retrieving the breast 11 repeatedly several times, the breast 11 is well massaged and becomes healthier.

Please refer to FIG. C3, which shows the control panel to control the timing of the whole schedule for breast augmentation. The timer knob sets up the time of the schedule for the therapy. The suction knob sets up the scale of the negative pressure applied to the breast 11. The release knob sets up the scale of the releasing of the negative pressure. The intensity knob sets up the biggest negative pressure in the whole schedule. The vacuum gauge shows the scale of the negative pressure. The pressure gauge shows the pressure inside the reservoir 7(FIG. A2). The knob “Power” is a switch for conducting a electric power. The knob “Full range/Bio circular” is a switch for setting up the condition of suction or the vibration. The sign “Sucker” is a communicating opening for connecting the shield 3 with vacuum generator 1. The sign “Supply” is a communicating tube to the shield 3 and the reservoir 7. The panel further has timer and controller for many kinds of setting.

Many setting are adjustable by the conditions of the breast, such as scale of the negative pressure, the timing for generating and releasing the negative pressure, and the timing for filling the filling medium into the shield, accordingly, a more efficient and safer breast augmentation can be achieved.

In the present invention, the reservoir 7 is filled with the curative filling medium 70 for replacing the air in the shield 3 so as to achieve a special medical effect. The filling medium is supplied from a tank or a pipeline.

Please refer to FIG. C4, which shows a conventional breast augmentation device with a manual air pump covering on a breast. Please refer to FIGS. C6-1 and C6-2. The breast augmentation device with a manual air pump further communicates to a reservoir 7 which provides a clean gas or liquid flowed into the shield 3. When the shield 3 has negative pressure generated therein, the filling medium 70 flows into the shield 3 to decrease the negative pressure inside the shield 3. In the FIG. C6-1, the reservoir 7 directly communicates with the shield 3. In FIG. C6-2, the reservoir 7 and the manual air pump both communicate with the shield 3 through the combination tube 4″.

Please refer to FIG. C5, which shows a conventional electrical breast augmentation device. Please refer to FIGS. C7-1 and C7-2, which shows an electrical breast augmentation device connecting with a reservoir 7. The reservoir 7 provides the filling medium 70 for the shield 3. When the shield 3 has negative pressure generated therein, the filling medium flows into the shield 3 to decrease the negative pressure inside the shield 3 and then the breast retrieves to the original shape. In the FIG. C7-1, the reservoir 7 directly communicates with the shield 3. In FIG. C7-2, the reservoir 7 and the electrical air pump both communicate with the shield 3 through the combination tube 4″.

Please refer to the FIG. C8, which shows various sizes of shields. Shield 81 shows the conventional shield for cupping therapy. The shield 81 is a shield having only one opening. The shields 82, 83 and 84 are shields having two openings. One of the openings is for suction. The other is for air reflux to reduce the negative pressure, or filling the filling medium into the shield to reduce the negative pressure. Furthermore, the shield could connect with a vibrator 88 to enhance the effect of breast augmentation.

The biggest difference between the present invention and the prior art is that the present invention further provides a reservoir 7 to provide a clean air or a filling medium 70, such as clean air or specific fluid, which flows into the shield 3. Another difference is that the shield 3 of the present invention has two openings. One communicates with the vacuum generator 1, and the other communicates with the reservoir 7. Therefore, the present invention is more efficient and safer for breast augmentation.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A device for sucking a body, comprising:
a vacuum generator for providing a negative pressure;
a reservoir for providing a filling medium stored therein;
a shield for covering an area of said body;
a first tube communicating said shield with said vacuum generator; and
a second tube communicating said shield with said reservoir.

2. The device according to claim 1, wherein said shield further comprises at least one opening for communicating said first and second tube.

3. The device according to claim 1, wherein said first tube and said second tube are combined as a combination tube to be connected to said at least one opening.

4. The device according to claim 1, wherein said first tube and said second tube are communicated with said shield through a connector.

5. The device according to claim 1, wherein said vacuum generator is selected from a group consisting of a manual air pump, an electrical air pump, and a vacuum pump.

6. The device according to claim 1, wherein said device has multiple vacuum generators.

7. The device according to claim 1, wherein said reservoir has a valve for controlling a communication of said second tube.

8. The device according to claim 1, wherein said first tube connects to a controller for controlling timings of a setup, a generation, and a release of said negative pressure.

9. The device according to claim 1, wherein said filling medium is one of a gas and a liquid.

10. The device according to claim 1, wherein said filling medium is provided from one of a can and a supplying tube.

11. The device according to claim 1, wherein said shield is selected from various standards.

12. The device according to claim 2, wherein a number of said opening is one of one and two.

13. The device according to claim 1, wherein said shield further comprises a vibrating mechanism.

14. A method for sucking a body through a sucking device, wherein said sucking device comprises a vacuum generator, a reservoir, a shield having at least one opening, a first tube and a second tube, said first tube communicates said shield with said vacuum generator and said second tube communicates said shield with said reservoir, said method comprising steps of:
(a) covering said shield on an area of said body;
(b) generating a negative pressure in said shield by said vacuum generator so as to gradually bulge said body area; and
(c) filling a filling medium into said shield from said reservoir for decreasing said negative pressure so as to restore said body area.

15. The method according to claim 14, wherein said wherein said first tube and said second tube arc communicating with said at least one opening.

16. The method according to claim 14, wherein said vacuum generator is selected from a group consisting of a manual air pump, an electrical air pump, and a vacuum pump.

17. The method according to claim 14, wherein said first tube further connects to a controller for controlling timings of a setup, a generation, and a release of said negative pressure.

18. The method according to claim 14, wherein said filling medium is one of a gas, and a liquid.

19. The method according to claim 14, wherein a number of said opening is one of one and two.

20. The method according to claim 14, wherein further comprising a stop of vibrating said shield by a vibrating mechanism connected thereon.
